(19) United States
(12) Patent Application Publication
(10) Pub. No.: US 2010/0145146 A1
(43) Pub. Date: Jun. 10, 2010

(54) ENDOSCOPIC DIGITAL RECORDING SYSTEM WITH REMOVABLE SCREEN AND STORAGE DEVICE

(75) Inventor: Patrick C. Melder, Marietta, GA (US)

Correspondence Address: PATZIK, FRANK & SAMOTNY LTD., 150 SOUTH WACKER DRIVE, SUITE 1500, CHICAGO, IL, 60606, US

(73) Assignee: ENVISIONIER MEDICAL TECHNOLOGIES, INC., Woodstock, GA (US)

(21) Appl. No.: 12/709,900

(22) Filed: Feb. 22, 2010

Related U.S. Application Data
(63) Continuation-in-part of application No. 29/322,352, filed on Aug. 1, 2008, now Pat. No. D611142.
Application No. 29/322,352 is a continuation-in-part of application No. 29/322,349, filed on Aug. 1, 2008, now Pat. No. D610601.
Application No. 29/322,349 is a continuation-in-part of application No. 29/322,353, filed on Aug. 1, 2008, now Pat. No. D611520.
Application No. 29/322,353 is a continuation-in-part of application No. 11/768,965, filed on Jun. 27, 2007.
Application No. 11/768,965 is a continuation-in-part of application No. PCT/US2005/047281, filed on Dec. 28, 2005.
(60) Provisional application No. 61/248,443, filed on Oct. 3, 2009.

(51) Int. Cl. A61B 1/04

(52) U.S. Cl. 600/112

(57) ABSTRACT

A medical imaging recording and viewing system adapted for use interchangably with a variety of endoscopes. The system includes a portable hand-held device that may be removably placed within a cradle affixed to the endoscope. An interface between an image acquisition device of the endoscope and the portable device permits images to be transmitted to and stored within the portable device. The portable device also may include a viewing screen to permit a user to view the images. A docking station may receive the portable device for transferring images to a separate viewing device and for recharging the battery of the portable device.

### BACKGROUND OF THE INVENTION

This application claims the benefit of U.S. Provisional Application No. 61/248,443, filed Oct. 3, 2009, and is a continuation in part of U.S. Design Patent Application No. 29/322,352, filed Aug. 1, 2008, U.S. Design Patent Application No. 29/322,349, filed Aug. 1, 2008, U.S. Design Patent Application No. 29/322,353, filed Aug. 1, 2008, and U.S. patent application Ser. No. 11/768,965, filed Jun. 27, 2007 which is a continuation-in-part of International Patent Application No. PCT/US2005/047281, filed Dec. 28, 2005 and which claims the benefit of U.S. Provisional Application No. 60/639,451, filed Dec. 28, 2004, the entirety of all of these applications are hereby incorporated by reference.

### FIELD OF THE INVENTION

This invention relates generally to portable, hand-held endoscopic recording systems, and more particularly to a digital endoscopic recording system having a docking station with a removable LCD screen with internal memory for storage and reviewing endoscopic images.

### BACKGROUND ART

As is well known, various technologies are available to the medical profession for use in viewing and imaging internal organs and systems of the human body. For example, otolaryngologists often require an endoscopic examination of the patient's upper respiratory system. One of the most common tools used by otolaryngologists to view the upper respiratory system is an endoscope. Similarly, endoscopes are used by surgeons and physicians in many fields of medicine in order to view parts of the human body internally for examination, diagnosis, and treatment. Initially, endoscopes included only an eyepiece, through which the physician could view the area being examined and/or treated. However, modernization of medical tools have produced more modern endoscopic systems that include camera assemblies with a camera head attached to the proximal end or eyepiece of the endoscope, typically via a coupler. For example, U.S. Pat. No. 4,697,894 issued to Takamura et al and discloses a connection device for connecting an associated unit to an eyepiece section of an endoscope. U.S. Pat. No. 4,697,894 is incorporated herein by reference.

Visual documentation is important in medicine, particularly for improved patient care and educational and training purposes. There are several variations of camera systems available to physicians that attach to the endoscope for imaging what the endoscope is viewing. For still photography a 35 mm analog single lens reflex (SLR) camera or a modern digital (SLR and non-SLR) camera can be used. For video photography a camera head, camera control unit, adapters to fit the endoscope onto the camera, and a video system monitor are used for viewing. All methods of endoscopy require a light source for illumination. These systems are commonly used in doctor's offices, emergency rooms, hospital rooms, and operating rooms, but are very expensive, not easily adapted, and not configured to be easily transported between and among multiple locations.

The cameras currently available to medical professionals are not easily configured for endoscopic imaging. A camera control unit is required to control the camera and process the digital or analog image signals received by the camera from the endoscope. Analog images are processed through an analog/digital converter and transmitted as a digital image. Digital images are captured directly to a charged coupled device (CCD) that captures images in pixel format as an electrical charge. This information is then processed with a varying array of filters to produce color images. The images must then be transmitted to a computing device for storage, editing, and further processing of the data.

The camera control unit and accompanying computer and viewing screen are bulky, heavy, and not easily transported to different locations. In addition to the size and transport limitations, the systems currently available can range in cost from $10,000 or more for just the camera and camera control unit. In addition to the cost of the camera and camera control unit, the endoscope, and typically a light source must be purchased.

Manufacturers have attempted to produce digital archiving platforms to allow easy integration into the digital age by integrating disc burners and hard drives into the endoscopy units so that exams can be stored directly onto removable media. These alternatives, however, limit editing of the images and are not very dynamic. Other manufacturers have attempted to produce endoscopy units that capture the images directly into a proprietary computer system designed for the specific function of video capturing and archiving. These systems provide better data manipulation, but can cost more than $20,000, and thus not affordable for a small or cost-limited practice.

Some alternative systems have been designed with portable components. These portable component systems are smaller in size than the fixed systems, but still require a camera control unit, a monitor, a means for capturing the images, and a light source in addition to the main components of a camera and endoscope. Although these systems are classified as portable, they are heavy, cumbersome, and expensive. U.S. Pat. No. 6,432,046 issued to Yarush et al and discloses a hand-held portable camera for producing video images of an object, and has as an object to provide a camera which features a lighting system capable of high-intensity illumination without creating an over abundance of heat. Yarush et al discloses a fixed lens tube which receives a variety of apparently custom probes and, in certain embodiments, further requires one of several adapters to receive certain probes. Additionally, this aforementioned patent is not readily adapted to the standard fittings of the eyepiece of endoscopes used in medical practices.

Additionally, various technologies are available to the medical profession for use in recording and viewing endoscopic images. In the past, images were recorded using analog devices such as VHS recorders and still images were printed using printers. While analog devices recorded images, their use came with several limitations including, but not limited to, the inability to easily incorporate into electronic media for sharing and reviewing (like e-mail, electronic medical records, and PowerPoint®) the difficulty in cataloging analog images.

Newer means to store images include digital recorders. Many of these systems record to optical media such as DVDs and CD, while other devices record to flash RAM devices like USB drives. However, these digital systems are extremely bulky and are part of a larger component cart system not allowing for portability or use with multiple devices. Further, current digital systems do not allow for easy incorporation of electronic medical record data into the device to streamline data management.

### SUMMARY OF THE INVENTION

One aspect of present invention comprises an endoscopic imaging system which overcomes the foregoing and other difficulties. In one embodiment, the present invention provides a portable hand-held endoscopy system adapted for interchangeable use with a variety of endoscopes. The system includes an endoscope having a first end and a second end, the first end having an eyepiece and the second end having a viewing end, a battery operated digital camera having an optical input, viewing screen, digital signal processor and memory with embedded software for processing data from the processor and for displaying an image on the viewing screen, and a coupler having a first end and a second end, wherein the first end includes a connector for removably connecting to the eyepiece, and the second end includes a connector for coupling to the optical input of the digital camera. In one embodiment, the camera is a high definition HD endoscopic camera.

In another embodiment, the system may not include the viewing screen. Alternatively, or in addition, the system may include features to provide a high speed digital data transfer protocol port for coupling to an external device, such as a personal computing device.

In another embodiment, the present invention provides a portable hand-held endoscopy system adapted for interchangeable use with a variety of endoscopes. The system having an endoscope having a first end and a second end, the first end having an eyepiece and the second end having a viewing end. The system further comprising a battery operated unitary digital camera having an optical input, viewing screen, digital signal processor, memory with embedded software for processing data from the processor and for displaying an image on the viewing screen, and a coupler located at the distal end of the camera for removably connecting to the eyepiece of an endoscope.

In another embodiment, the distal end of the endoscope may include a charge coupled device or similar device for obtaining the image. The output of the charge coupled device is coupled to the camera.

In accordance with the more specific aspects of the present invention, one embodiment includes a high speed data transfer component (IEEE 1394, Wireless HD, 3G, 4G and similar cellular data transfer methods, USB and similar methods), which connects directly to the camera for sending signals from the camera to a personal computing device, removable data storage card and/or onboard mini hard-drive or flash memory and onboard controls, enabling more detailed camera control and image manipulation. Another embodiment of the present invention comprises a digital camera equipped with an LCD or similar screen for viewing the images, embedded software and one-touch controls for enhancing, manipulating and editing the images, and a media storage card for storing the images.

The invention described herein requires only a limited number of components, providing physicians with a portable, versatile, and less expensive system for endoscopic examinations and recording, archiving and sharing the images and videos thereof. Among other things, the present invention eliminates the need for accessory equipment that is infrequently used (e.g., VHS recorders, printers, CD/DVD burners). The system is easily transported to multiple locations, enabling healthcare providers greater versatility in the applications of endoscopic examinations and flexibility of the locations at which they examine patients. In addition to cost savings and flexibility, the high speed data transfer technology facilitates higher speed, lower cost data translation and manipulation, enhancing and expanding the quality of visual documentation generated without the need for special or costly computer systems.

The present invention is also a great teaching tool for residents in an academic environment. For patients and parents alike, the present invention provides an unparalleled ability to demonstrate pathology at the point of care. For an ever increasingly demanding, consumer driven patient, the present invention provides information at the point of care to allow the surgeon to breakdown the “credibility barrier.”

In another embodiment, the recording and viewing system of the present invention includes a portable hand-held device or image viewer (LCD and image processor) that may be removably placed within a cradle affixed to an endoscope, such as a distal chip endoscope. An interface between the camera or image acquisition device of the endoscope and the portable device permits the images (e.g., still shots and videos) to be transmitted and stored in memory on the portable device. A docking station may receive the portable image viewer for transferring images to a separate viewing device and for recharging the battery of the portable device.

In a preferred embodiment, the portable LCD device of the present invention (sometimes referred to as “the eGo”) is a mass storage device with full multimedia playback that can be mounted on flexible scopes or other mounts to encourage natural line of site aligned with the viewing field. On board data management and integration with EMRs eliminates frustrating and cumbersome tasks all too often associated with endoscopic data management.

The portable device may include a viewing screen to permit a user to view or replay images in the form of, for example, still shots or videos, on the portable device. The viewing screen may also be a touch screen to allow for a user to operate the portable device or input information and data into the portable device. For example, using the portable device LCD touch screen allows the user to tag an endoscopic exam to the patient's information without redundant effort. A gravity detecting system may also be used with the portable device to align the still shots and videos during playback and lighting control is optimized for any environment.

LED lighting technology is provided for brighter lighting capability than in many tabletop Halogen or Metal Halide light sources. Combining the present invention with high powered LED light source provides a convenient, versatile, and scalable endoscopic imaging system for use in multiple exam rooms in an outpatient clinical setting. This combination is also perfect for academic teaching environments and remote endoscopic diagnosis.

The portable device may also securely exchange data with either a network computer or electronic medical records system in a secure HL7 format and may include the surgeon's schedule. The data between the endoscopic camera, PC, and a web-storage data base is preferably synchronized, e.g., by docking the portable device into the docking station.

The system may also utilize a web-based storage and communication system which allows the endoscopist to securely access and share the endoscopic video and still shots. The web-based solution can be used with any web browser on any computer. Once videos are uploaded, they may be converted to flash (or similar) format for viewing via a web browser. The original videos are stored in a cloud server for full access, download, and editing at anytime, anywhere.

Other objects, features and advantages of the invention will be apparent from the following detailed disclosure, taken in conjunction with the accompanying sheets of drawings, wherein like reference numerals refer to like parts.

### BRIEF DESCRIPTION OF THE DRAWINGS

FIG. 1 is a perspective view illustrating one embodiment of the present invention in use;

FIG. 2 is a perspective view of another embodiment of the present invention;

FIG. 3 is perspective view of yet another embodiment of the present invention;

FIG. 4 is perspective view of yet another embodiment of the present invention;

FIG. 5 is a perspective view of another embodiment wherein a camera and coupler are provided as a unitary component, and includes a viewing screen;

FIG. 6 is a perspective view of another embodiment wherein a camera and coupler are provided as a unitary component, and includes a high speed data transfer port;

FIG. 7 is a perspective view of another embodiment wherein a camera and coupler are provided as a unitary component, and includes a swivel orientation adjuster;

FIG. 8 shows the camera unit of FIG. 7 but with the camera unit adjusted to different orientation;

FIG. 9 is a perspective view of another embodiment wherein a camera and coupler are provided as a unitary component, including a movable viewing screen and a swivel orientation adjuster;

FIGS. 10 and 11 are perspective views of a further embodiment wherein a camera and coupler are provided as a unitary component, including a swivel orientation adjuster;

FIG. 12 is a schematic of one embodiment of the unitary component;

FIG. 13 is a functional block diagram of one embodiment of the present invention;

FIG. 14 is a schematic of one embodiment of the present invention with the charge coupled device at the distal end of the endoscope;

FIG. 15 is a perspective view of yet another embodiment of the present invention;

FIG. 16 is an elevated, left side view of the embodiment of FIG. 15;

FIG. 17 is a top plan view of the embodiment of FIG. 15;

FIG. 18 is an elevated, front view of the embodiment of FIG. 15;

FIG. 19 is a bottom view of the embodiment of FIG. 15;

FIG. 20 is a perspective view of the embodiment of FIG. 15, partially in section;

FIG. 21 is an elevated, left side view of the embodiment of FIG. 15, partially in section, with the display portion omitted for clarity;

FIG. 22 is an enlarged, bottom perspective view of the display post connector of the embodiment of FIG. 15;

FIG. 23 is an enlarged, top perspective view of the display post connector of the embodiment of FIG. 15;

FIG. 24 is an enlarged, bottom perspective view of the display rotational connector of the embodiment of FIG. 15;

FIG. 25 is an enlarged, top perspective view of the display rotational connector of the embodiment of FIG. 15;

FIG. 26 is a perspective view of the embodiment of FIG. 15 showing, in particular, the camera connected to an external light source and a rigid endoscope, and being held in a pistol grip manner;

FIG. 27 is a perspective view of the embodiment of FIG. 15 showing, in particular, the camera connected to an external light source and a flexible endoscope, and being held in a top, distal tip control manner; and

FIG. 28 is a perspective view of the embodiment of FIG. 15 showing, in particular, the camera connected to an external light source and a rigid endoscope, and being held in a bottom, distal tip control manner.

FIG. 29 is a perspective view of one embodiment of an endoscopic digital recording system with a removable screen.

FIG. 30 is a rear elevated view of the embodiment of the endoscopic digital recording system of FIG. 29.

FIG. 31 is a front elevated view of the embodiment of the removable screen of FIG. 29.

FIG. 32 is a bottom plan view of the embodiment of the holder of FIG. 29.

FIG. 33 is a perspective view of one embodiment of a remote trigger endoscopic image recording and viewing system.

FIG. 34 is a perspective view of one embodiment of a distal chip endoscopic image recording and viewing system.

FIG. 35 is a perspective view of one embodiment of a optical rigid scope image recording and viewing system.

FIG. 36 is a schematic block diagram of one embodiment of the portable device of the present invention, and includes a digital signal processing device such as a DM355.

FIG. 37 is a front perspective view of an embodiment of a docking station for a removable screen.

FIG. 38 is a perspective view of another embodiment of a docking station for a removable screen.

FIG. 39 is a schematic illustration of the communication channels between the hand-held camera and portable device and storage devices and other systems.

FIG. 40 is a flow chart depicting migration and integration of data from the endoscopic digital recording system with removable screen and storage to Web Services API, streaming media to cloud storage, and online collaborative site API.

FIG. 41 is a schematic block diagram of one embodiment of a docking station.

FIG. 42 is a schematic block diagram of one embodiment of the portable hand held camera.

### DETAILED DESCRIPTION OF THE INVENTION

While this invention is susceptible of embodiment in many different forms, there is shown in the drawings and will herein be described in detail several specific embodiments, with the understanding that the present disclosure is to be considered merely an exemplification of the principles of the invention and the application is limited only to the appended claims.

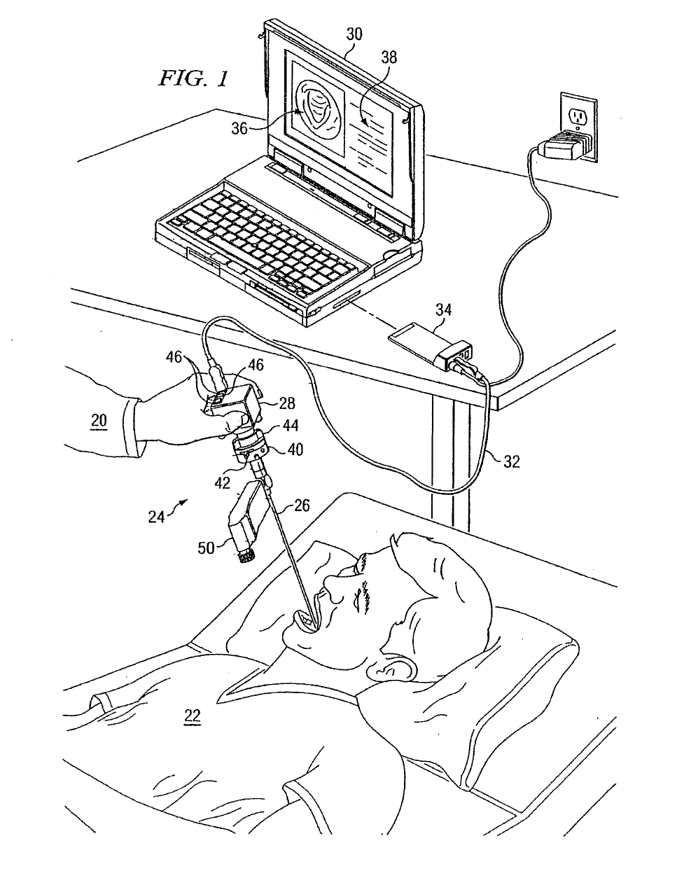

Referring to the Drawings, and particularly to FIGS. 1 and 2 thereof, there is shown an endoscopic imaging system comprising a first embodiment of the invention. Referring specifically to FIG. 1, a physician 20 is shown performing an endoscopic examination of a patient 22 using an endoscopic imaging system 24. An endoscope 26 is inserted into the patient 22. The images seen by the endoscope 26 are received into a portable endoscopic digital camera 28 capable of high speed data transfer and then transmitted to a computer 30 by means of a high speed data transfer connection cable 32, for example a USB cable. The cable 32 connects into a multifunctional interface card 34, which also supplies power to the camera 28. Alternately, the high speed data transfer connection cable can connect directly to a computer or similar computing device without a multifunctional interface card if said high speed data transfer connection is built into the computing device. Examples of the high speed data transfer consistent with the present invention include various protocols such as IEEE 1394, USB, BLUETOOTH and 80211.b (or similar wireless technology). As a further alternative, the camera 28 may be battery operated. The computer 30 enables the physician 20 to control the camera 28 and further manipulate the data received during the examination. The endoscopic images 36 are displayed on a computer screen 38. The computer screen 38 may either be an on-board screen on a notebook style computer, or an independent monitor networked with the computer 30, or a desktop workstation computer.

Referring specifically to FIG. 2, there is shown an enlarged view of the endoscopic imaging system 24 shown in FIG. 1. The endoscope 26 of FIG. 1 is constructed of a rigid material such as stainless steel, or other materials approved for use in medical applications. The endoscope 52 shown in FIG. 2 is constructed from a flexible material approved for use in medical applications. The endoscope 52 is coupled to a portable endoscopic digital camera 28 capable of high speed data transfer by a coupler 40. In one embodiment, the coupler 40 includes a standard C or C/S coupler and is equipped with a locking mechanism 42 which holds the endoscope 26 or 52 securely onto the camera 28. Alternately, there is no C or C/S coupler. The coupler 40 receives the images from the endoscope 52. The coupler 40 includes at least one lens assembly that moves to focus the image onto a transducer or other similar device internal to the camera 28. Additionally, the image coupler allows for zooming and magnification of the images. The coupler 40 couples to the camera 28 and a focus ring 44. The focus ring 44 assists the physician to focus the image to obtain a clearer, better quality image.

In one embodiment, the portable endoscopic digital camera 28 capable of high speed data transfer uses a single charged coupled device (CCD) (described further below) as the image acquisition device, together with a high speed data transfer input/output ports 46. Depending on the camera mode selected by the user at the computer 30, the portable endoscopic digital camera 28 transmits the images to the computer 30 where the images are viewed and stored. A digital camera, based on IEEE 1394 or the like, for use with the endoscopic imaging system 24 may also be equipped with a triple charged coupled device (CCD) and have multiple high speed data transfer input/output ports 46. The multiple high speed ports are beneficial because the additional throughput desired with the triple CCD. Additionally, the portable endoscopic digital camera capable of high speed data transfer may also be equipped with complimentary metal oxide semiconductors (CMOS) for image acquisition. The embodiment shown in FIGS. 1 and 2 include a light source 50 coupled to the endoscope 26, 52 and coupler 40. The light source 50 provides additional lighting for a better view of the area being examined by the endoscope 52. The light source 50 shown is battery operated. However, the light source may be operated by an external power source. Alternatively, an external light source and light guide cable may be provided as the light source. Still further, certain endoscopes are equipped with a light source.

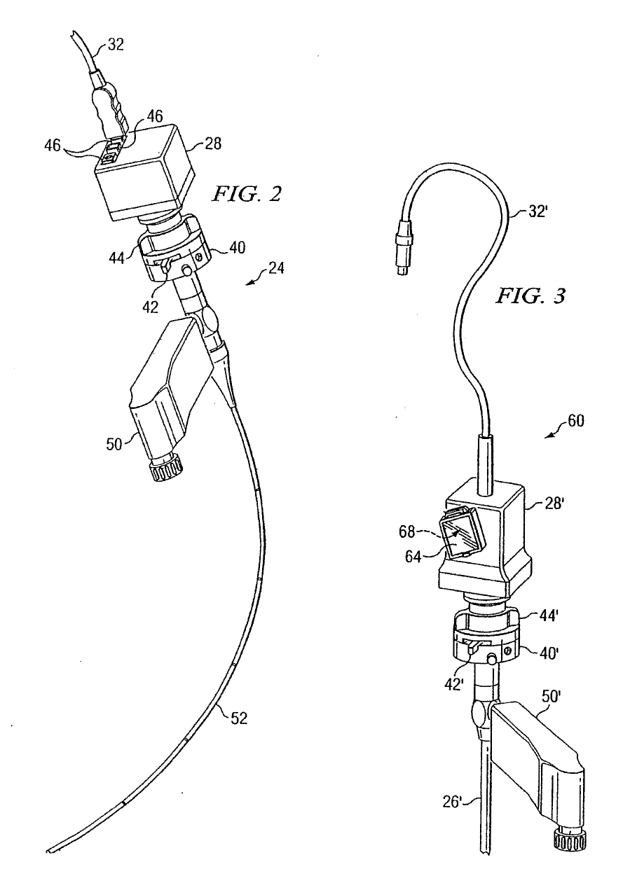

Referring now to FIG. 3, there is shown an endoscopic imaging system 60 comprising a second embodiment of the invention. Many of the component parts of the endoscopic imaging system 60 are substantially identical in construction and function to component parts of the endoscopic imaging system 24 illustrated in FIGS. 1 and 2 and described hereinabove in conjunction therewith. Such identical component parts are designated in FIG. 3 with the same reference numerals utilized above in the description of the endoscopic imaging system 24, but are differentiated there from by means of a prime (′) designation.

The endoscopic imaging system 60 differs from the endoscopic imaging system 24 of FIGS. 1 and 2 in that the endoscopic imaging system 60 includes a camera 28′ with an on-board LCD screen 64 and on-board one-touch camera controls with embedded software for manipulating, enhancing, and adjusting the data. It will be appreciated that the screen may be an LCD screen, LED screen or any other similar monitor. Installed into the camera 28′ is embedded memory in the form of a mini hard-drive or flash memory and/or a digital media storage card which stores the images from the endoscopic examination until the camera 28′ can be downloaded into a personal computing device through the high speed connection cable 32′. Such memory is described further below.

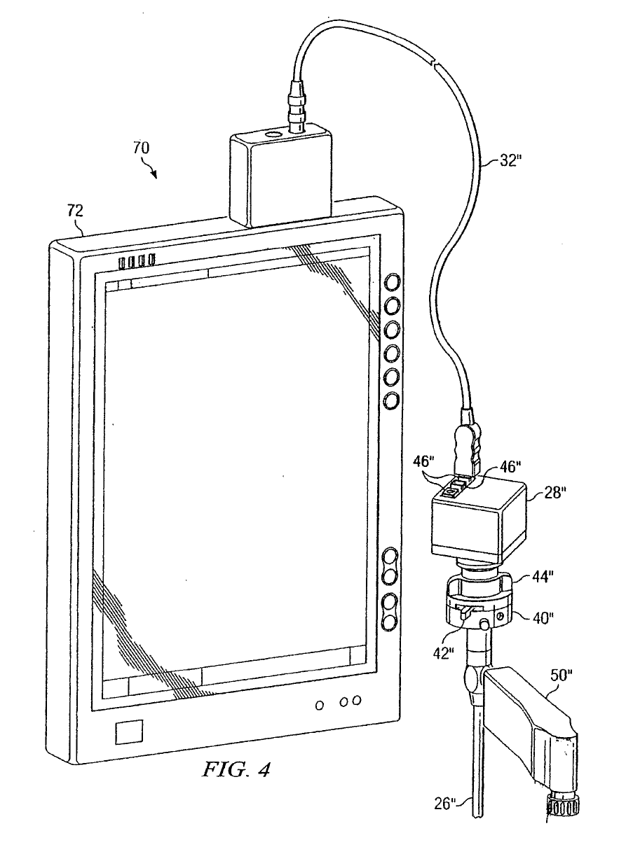

Referring now to FIG. 4, there is shown an endoscopic imaging system 70 comprising another embodiment of the invention. Many of the component parts of the endoscopic imaging system 70 are substantially identical in construction and function to component parts of the endoscopic imaging system 24 illustrated in FIGS. 1 and 2 and described hereinabove in conjunction therewith. Such identical component parts are designated in FIG. 4 with the same reference numerals utilized above in the description of the endoscopic imaging system 24, but are differentiated there from by means of a prime (″) designation.

The endoscopic imaging system 70 differs from the endoscopic imaging system 24 of FIGS. 1 and 2 in that the endoscopic imaging system 70 is equipped with a hand-held personal computing device 72. The hand-held personal computing device 72 may be an iPod® as manufactured by Apple Computer, Palm Pilot™, or other similar personal computing devices known to those skilled in the art.

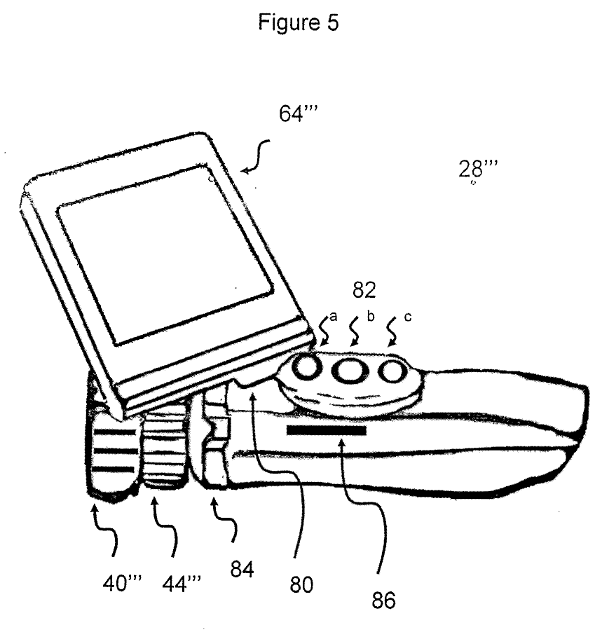

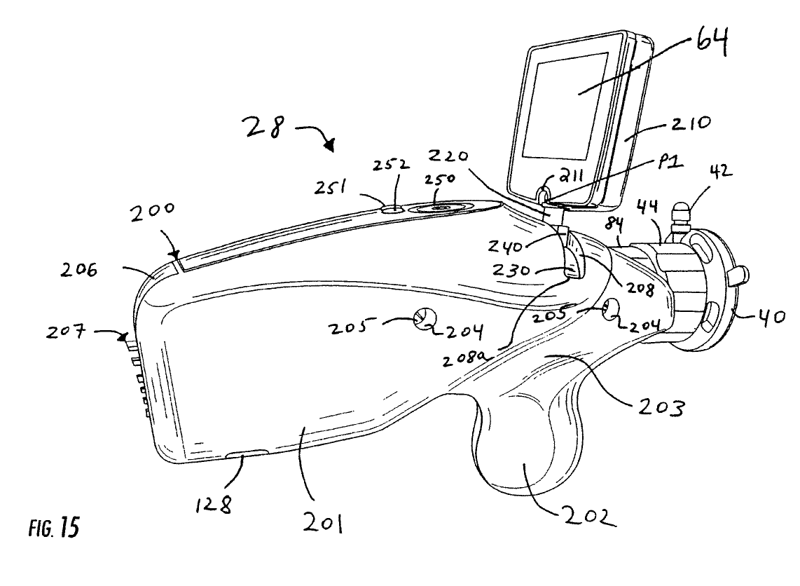

FIG. 5 is a perspective view of another embodiment wherein a camera 28′″ and coupler 40′″ are provided as a unitary component, including a viewing screen 64′″. The viewing screen may be a liquid crystal display (LCD) or thin film transistor (TFT) screen. The viewing screen is mounted to the unitary camera unit via a swivel pivot point hinge 80. The camera unit includes user input controls 82 (a,b,c) for manipulating, enhancing and adjusting data, via embedded software. The unitary camera unit also includes an endoscope coupler 40′″ for coupling to an endoscope such as that shown in FIG. 1-4. In addition, the unitary camera unit includes a focus ring 44 and a zoom ring 84 so that the physician may adjust the image for display on the viewing screen. However, the function of focus and zoom may also be accomplished via the embedded software and the user input controls 82. A slot 86 is provided for connection of a removable flash memory card (see FIG. 15). The removable flash memory card allows the data to be transferred to another device in order to view or store the data, as desired.

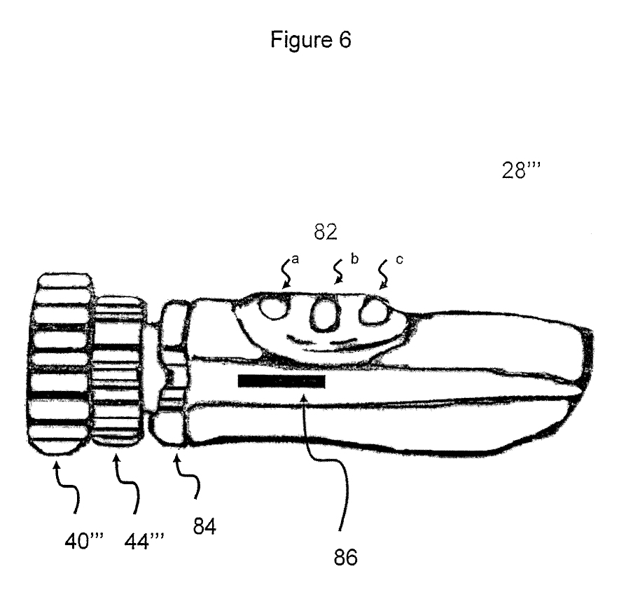

FIG. 6 is a perspective view of another embodiment wherein a camera 28′″ and coupler 40′″ are provided as a unitary component, including a high speed data transfer port 46 (not seen in FIG. 6). The high speed data transfer port 46 allows connection to the various external devices as noted above, such as a handheld PC device.

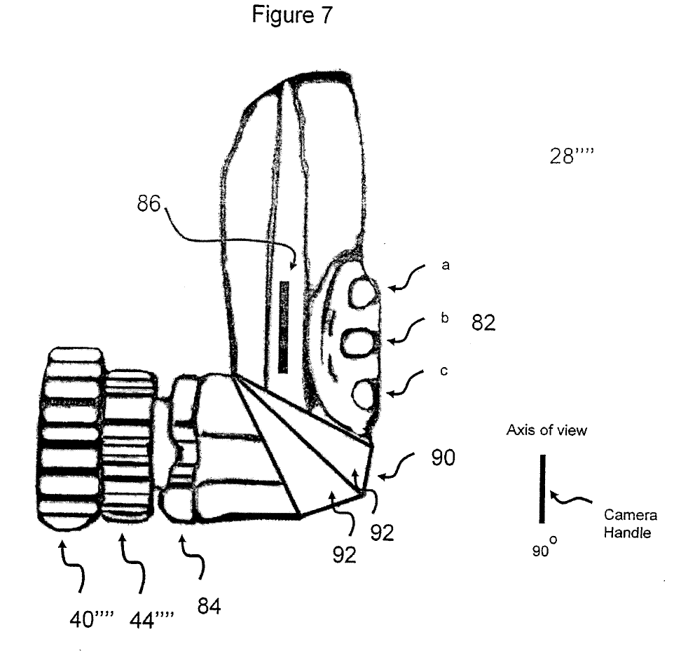

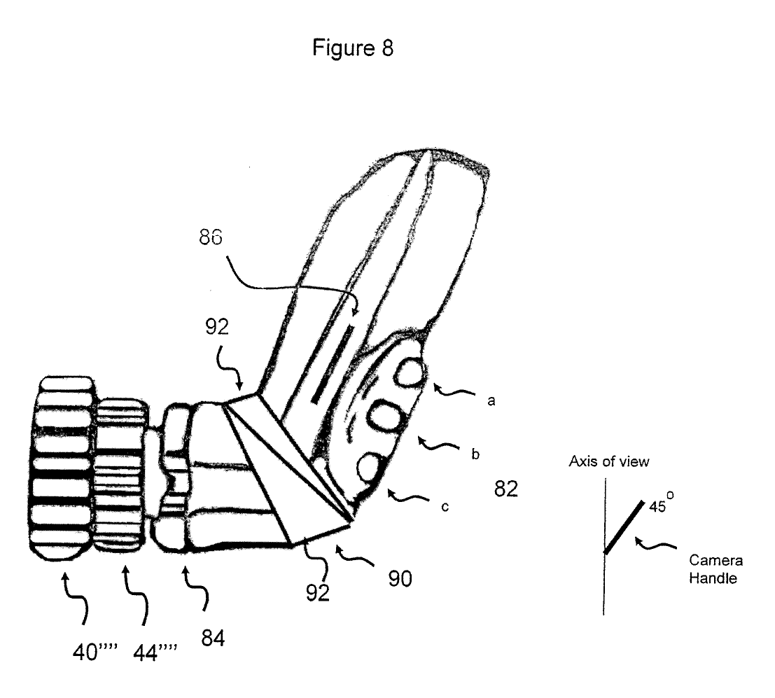

FIG. 7 is a perspective view of another embodiment wherein a camera 28″″ and coupler 40″″ are provided as a unitary component, including a swivel orientation adjuster or elbow joint 90. The swivel orientation adjuster 90, in one embodiment, is a series of one or more sleeves 92 having a certain profile such that twisting or rotating the camera unit allows the camera unit to assume various orientations, such as the one shown in FIG. 7 and in FIG. 8.

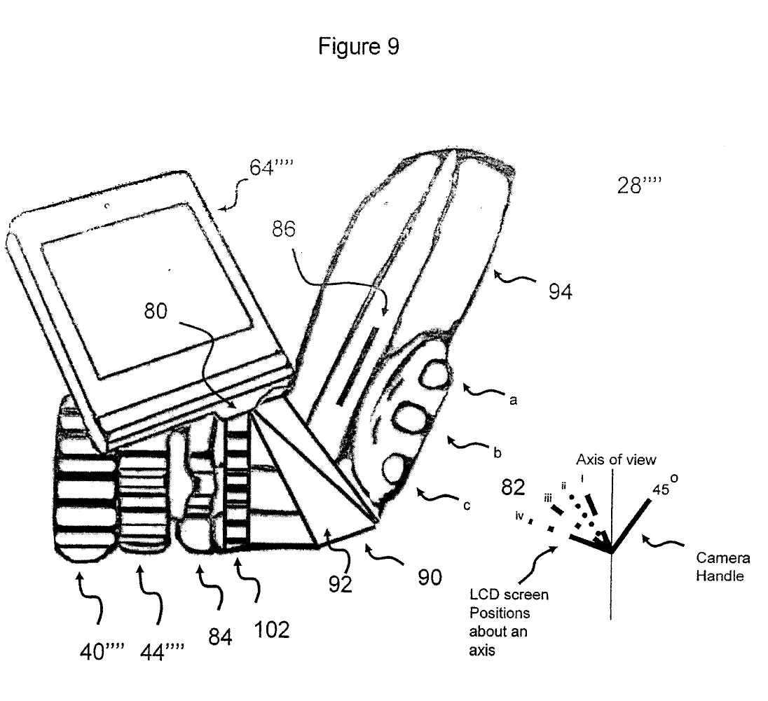

FIG. 9 is a perspective view of another embodiment wherein a camera 28″″ and coupler 40″″ are provided as a unitary component, including a hinged coupled flip viewing screen 64″″ and a swivel orientation adjuster 90. It will be appreciated from FIG. 9 that the handle or camera body 94 assumes one orientation with respect to the coupler portion 40″″ and the flip viewing screen 64″″ assumes another and independent orientation with respect to the coupler portion.

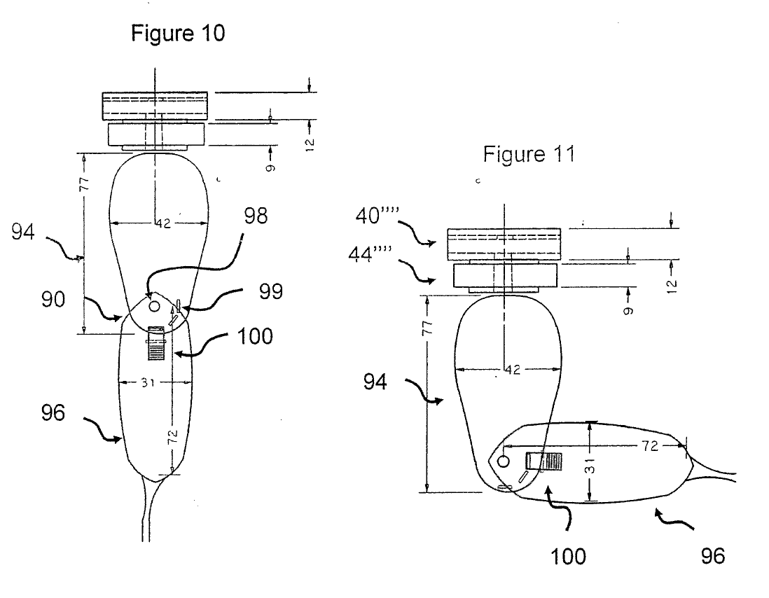

FIGS. 10 and 11 show a variation of the adjuster 90. In this instance, the camera body 94 is coupled to an extension portion 96 at a pivot point which includes a pin 98. The camera body 94 includes a plurality of recesses 99 which receive a detent or pawl 100 to lock the body 94 and extension portion 96 in position. FIG. 10 shows an in-line orientation, wherein FIG. 11 shows an offset orientation of 90 degrees.

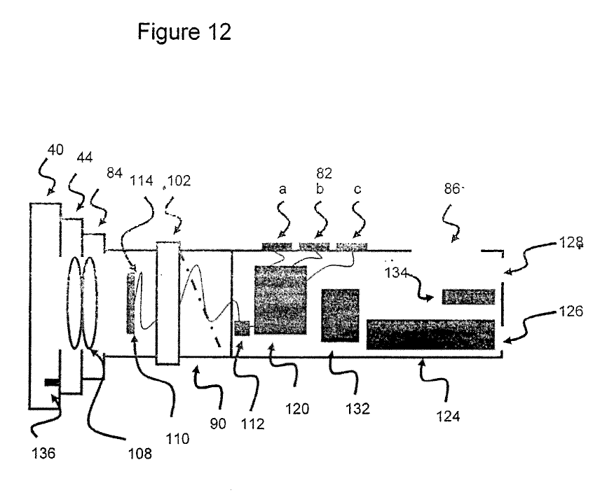

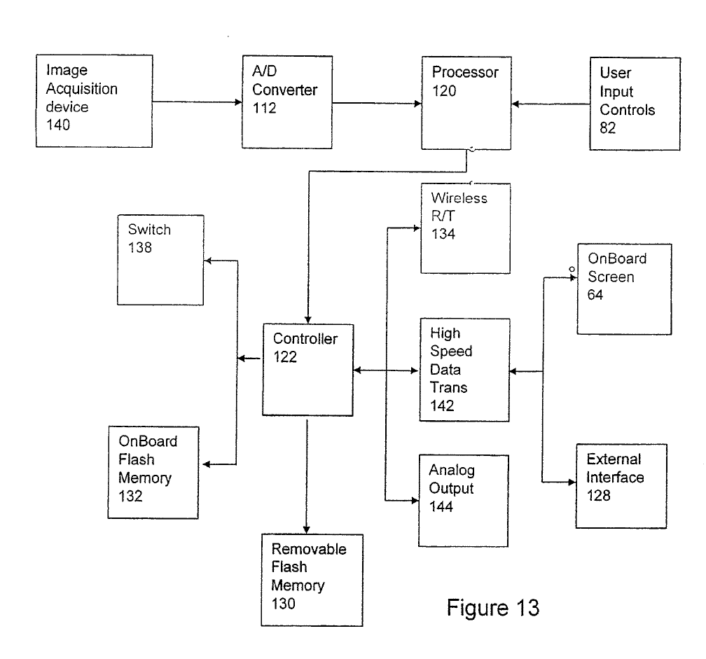

FIG. 12 is a schematic of one embodiment of the unitary component. The endoscope coupler 40 is shown adjacent to the focus ring 44 which is adjacent to the zoom ring 84. The zoom ring 84 is adjacent to the image ring 102. The elbow joint 90 is shown connected to the body 94. The distal end of the body 94 of the camera unit contains the optical lens mechanism 108 which accommodates the focus and zoom functions, in order to direct the image onto the CCD or CMOS chip 110. The chip 110 is connected to the input of an analog-to-digital converter 112 via the ribbon wire 114. The output of the A/D converter 112 is coupled to a digital signal processor/camera processor 120. The user input controls 82 are coupled to the processor 120, or alternatively to a controller 122 such as shown in FIG. 13. The proximal end of the body 94 also includes a battery 124 and a connector 126 for external DC. The proximal end of the body 94 also includes an I/O high speed data transfer port 128 and a connector 86 for a removable flash memory card 130 (see FIG. 13). The body 94 also includes on board flash memory 132. Finally, a wireless radio transceiver 134 is shown for wireless downloading of data and wireless control of the camera unit. A power on peg 136 is shown. The power on peg 136 includes a switch 138 (see FIG. 13) which is coupled to the controller 122. The memory 132 includes a code for a sleep mode and power up routine, or similar battery saving features. The unit is normally in a sleep mode as one skilled in the art will appreciate. Upon the coupler 40 engaging an endoscope, the power on peg 136 is engaged and the power up routine is initiated. As is shown, ribbon wire 114 or other conductors extend within the elbow joint 90 from the distal coupler end to proximal body.

FIG. 13 is a functional block diagram of one embodiment of the present invention. The image acquisition device 140 may be an CCD chip 110, for example. The high speed data transfer port 142 is shown coupled to the on board screen 64 and the port 128 for connection to an external device. An analog output 144, such as audio S, is provided for coupling to an external device.

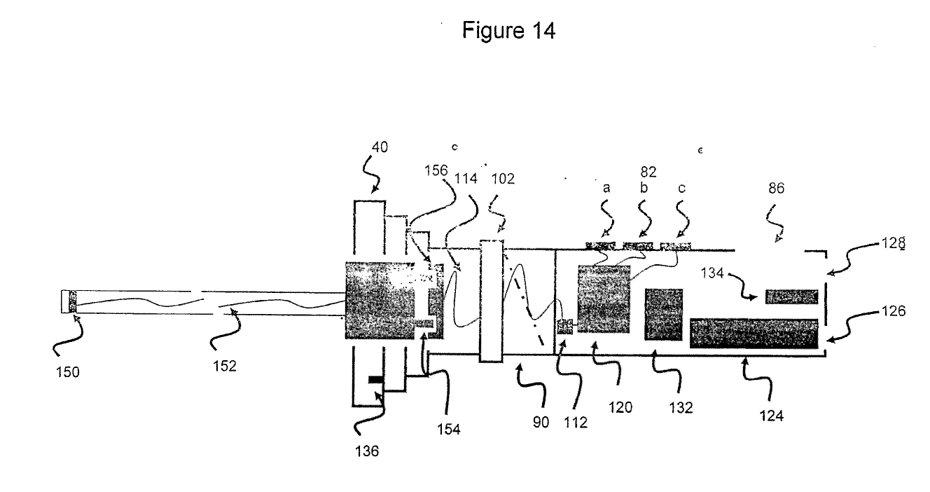

FIG. 14 is a functional block diagram of one embodiment of the present invention with the charge coupled device 150 or other image acquisition device at the distal end of the endoscope. Conductors 152 are coupled to the device 150 and extend within the endoscope. The coupler 40 includes an electrical and mechanical connector 154 for coupling to an electrical and mechanical connector 156 having the ribbon connector wire 114. The electrical and mechanical connector 154 is also adapted for coupling to the endoscope and the conductors 152. For example, the connector 154 may include a portion connected to the endoscope and a portion connected to the coupler 40. The system of FIG. 14 is otherwise similar to that shown in FIG. 12.

Another embodiment of the present endoscopic camera 28 is shown in FIGS. 15 through 28 as comprising camera housing 200, display housing 210 enclosing viewing screen or video display 64, coupler 40, focus ring 44, and zoom ring 84. Both camera housing 200 and display housing 210 are preferably constructed from high impact plastic, although other sturdy materials may alternatively be used. Camera housing 200 includes an elongated main body region 201, a bulbous gripping region 202, and two elongated, indented forefinger or index finger accepting regions 203 on opposing left and right sides of main body region 201, proximate bulbous gripping region 202. A high speed I/O data transfer port 128 permits both digital data transfers to an external computer, such as a personal desktop or laptop computer, via a conventional Universal Serial Bus (USB) interface, as well as analog video output to a conventional video display monitor, via an appropriate accessory AV cable. Data transfer port 128, when coupled to a PICTBRIDGE®-compatible USB printer, permits still images captured by the present endoscopic camera to be printed directly, without the need for an intermediate external computer. A power-on switch (not shown) is disposed on the left side of main body region 201.

A snap fit battery door 206, removable with the aid of a plurality of gripping ribs 207, permit access to a portion of the interior of camera housing 200, to permit removal and replacement of a rechargeable battery powering the endoscopic camera, as well as the insertion and removal of a flash memory card storing captured motion video and/or still images. A plurality of screw holes 205 and associated screws 204 permit camera housing 200 to be constructed from a plurality of housing portions.

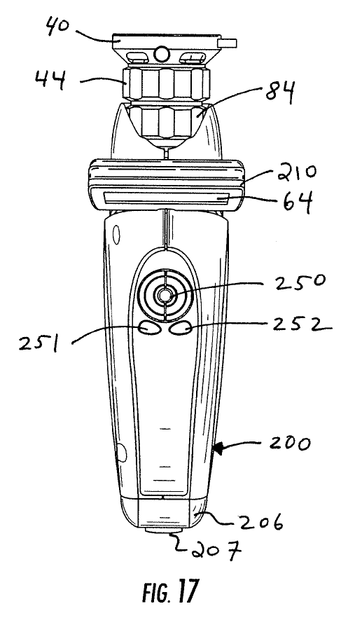

As best seen in FIGS. 17-19, a plurality of user input control switches are disposed on camera housing 200. In particular, the top surface of camera housing includes direction button 250, mode button 251, and menu button 252. Direction button 250 is preferably a digital joystick, normally spring biased to remain in a central, vertical orientation, which may be momentarily rocked into forward, reverse, left and right orientations, relative to its central orientation. One of the functions of direction button 250 is to select a digital zoom level for image viewing and capture. Movement of direction button to the forward or reverse orientation causes an associated positive or negative change in the digital zoom level of the image to be viewed and the still or motion video image to be captured. In a preferred embodiment, a zoom level of up to 4× digital magnification may be selected using direction button 250. Mode button 251 and menu button 252 are preferably pushbutton, momentary switches.

Direction button 250 performs several additional functions, in conjunction with mode button 251, menu button 252, and an on-screen menu presented to the physician using video display 64, under control of the microprocessor, or digital signal processor, contained within the housing of the present endoscopic camera. In particular, using these three buttons, the physician can play back video clips and select from amongst still images for viewing, view an index of “thumbnail” images of such recordings and still images, fast forward, fast reverse, and stop playing video clips, select a video/still capture image resolution mode of 1, 3 or 6 mega-pixels, record audio voice clips, turn image date stamping on and off, enable and disable automatic image stabilization, adjust the white balance setting of captured images, turn image histogram displays on and off, choose from amongst natural color, black and white, and sepia toned image capture, manually adjust the image exposure level, activate a 10-second electronic shutter self-timer, enable/disable on screen display icons, select the video output resolution (i.e., 640×480 or 320×240 pixels), and combine two images taken individually into one photograph.

Moreover, the on-screen menu may also be employed to delete images and video clips, view a “slide show” of previously captured images, and to print images directly to an attached, PICTBRIDGE®-compatible printer. In addition, the on-screen menu may be used to set an internal date and time, enable/disable audio beep sounds, set the display flicker frequency to 50 Hz or 60 Hz, set the direct, analog TV output of the high speed I/O data port to either NTSC or PAL video formats, set the brightness of video display 64, format the internal and removable storage media; turn automatic shutoff on and off, set the language for the on screen display, and set a mode of operation of the USB port (depending upon the setting, when connected to a personal computer via the high speed USB port, the on screen display will either display a menu permitting the physician to select a desired connection mode, will automatically connect in “removable disk” mode, or will automatically enter printer mode).

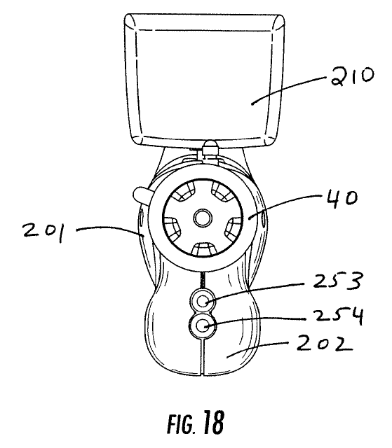

In addition to direction button 250, mode button 251, and menu button 250, as best seen in FIGS. 18 and 19, the present endoscopic camera further includes two redundant motion video record buttons 253, 253a; and two redundant still photograph shutter buttons 254, 254a, all comprising momentary, pushbutton switches and disposed on camera housing 200. In particular, either video record button 253 or 253a may be depressed to record a video clip, and either still photograph shutter button 254 or 254a may be depressed to capture a still photograph. As shown in FIG. 18, a first grouping of video record and still photograph shutter buttons, video record button 253 and still photograph shutter button 254, are disposed on a front surface of bulbous gripping region 202. As shown in FIG. 19, a second grouping of video record and still photograph shutter buttons, video record button 253a and still photograph shutter button 254a, are disposed on a bottom surface of elongated main body region 201, behind bulbous gripping region 202.

The provision of redundant video record and still photograph shutter buttons serve to facilitate ease of operation by the physician in recording video clips and still images using the present endoscopic camera. In particular, depending upon the type of endoscope attached to the present endoscopic camera, as well as the type of endoscopic inspection being performed, it may be convenient for the physician to hold the present endoscopic camera in a variety of different manners. Depending upon the physician's particular orientation and grip of the present endoscopic camera and attached endoscope, it may be more convenient in some circumstances to use buttons 253 and/or 254; and in other instances to instead use buttons 253a and/or 254a.

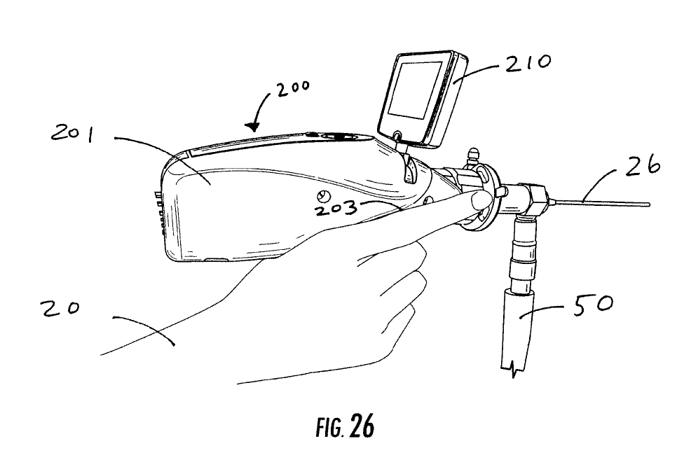

Several different ways in which the present endoscopic camera and an attached endoscope may be held by the physician are shown in FIGS. 26-28. In FIG. 26, the physician 20 is shown holding camera housing 200 in a right-handed, pistol grip fashion whilst performing otolaryngology using a rigid endoscope 26 and an external light source 50. As shown in FIG. 26, the physician's middle, ring and little fingers of the right hand are all wrapped around the bulbous gripping region of the camera housing. This bulbous gripping region may be substantially bulbous or bulb-like, substantially spherical, substantially spheroidal, substantially oblate spheroidal, or substantially ellipsoid in shape. The physician's forefinger, or index finger, is placed along elongated indented forefinger accepting region 203 on the right-hand side of camera body 201. When held in this manner, it will be more convenient for the physician to record video clips and still images using video record button 253 and still photograph shutter button 254, respectively, by using the middle or ring finger to depress the desired button in a trigger-like manner. Accordingly, as best seen in FIG. 26, housing 200, including the combination of bulbous gripping region 202 (obscured by the physician's hand in this figure), forefinger accepting region 203, and buttons 253 and 254, provide a highly ergonomic means of holding the present endoscopic camera. Moreover, inasmuch as two, substantially identical forefinger accepting regions 203 are symmetrically disposed on opposing sides of camera housing 200, and inasmuch as buttons 253 and 254 are disposed proximate a front, central region of bulbous gripping region 202, the present endoscopic camera may be held and operated in a pistol grip manner using either the left hand or the right hand.

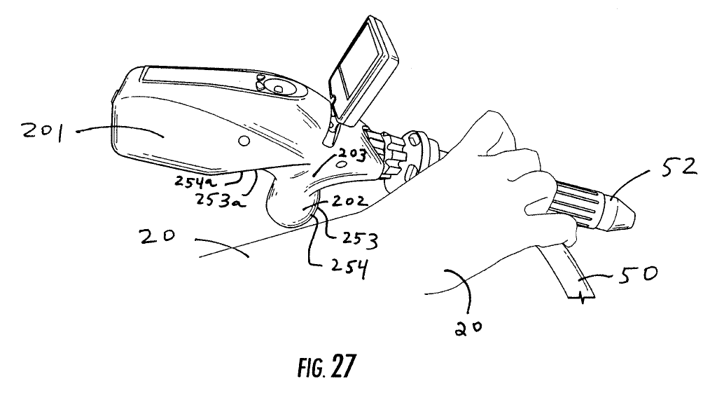

Another manner of gripping the present endoscopic camera, in conjunction with an attached flexible endoscope 52, is shown in FIG. 27. As shown, the physician 20 is holding the flexible endoscope in a top, distal tip control manner. When so held, the physician has relatively easy access to either buttons 253 and 254, or buttons 253a and 254a, and can use whichever is considered by the physician to be most convenient.

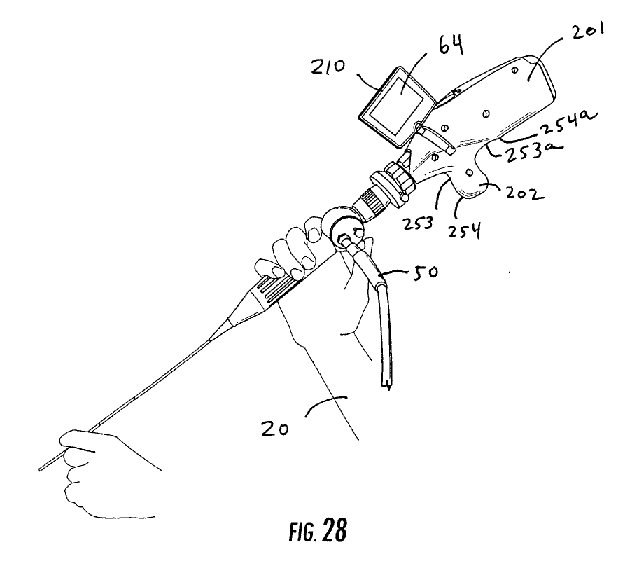

Yet another manner of gripping the present endoscopic camera, in conjunction with the use of a flexible endoscope, is shown in FIG. 28. As shown, the physician 20 is holding the flexible endoscope in a bottom, distal tip control manner. Again, the he physician has relatively easy access to either buttons 253 and 254, or buttons 253a and 254a, and can use whichever is considered to be most convenient.

Still another manner of gripping the present endoscopic camera, not shown in the drawings, is for the physician to “palm” the elongated main body region with an overhand grip. When so held, it is generally more convenient for the physician to depress, or squeeze, buttons 253a and/or 254a, on the underside of the camera housing, to record a video clip or take a still photograph, respectively.

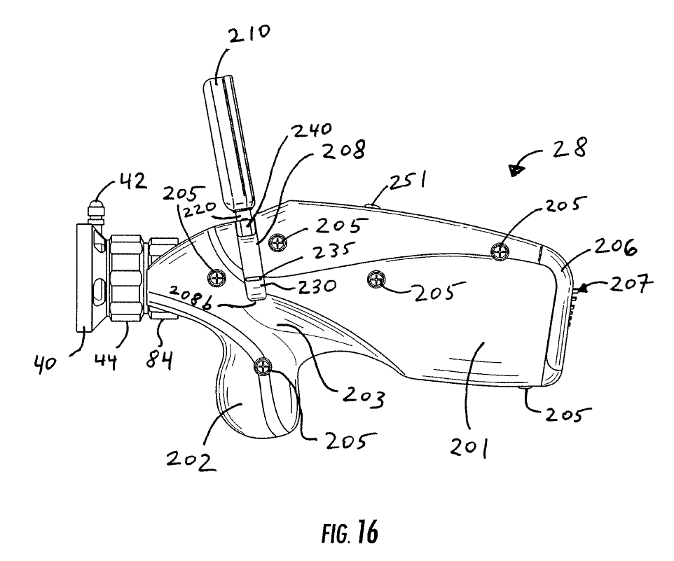

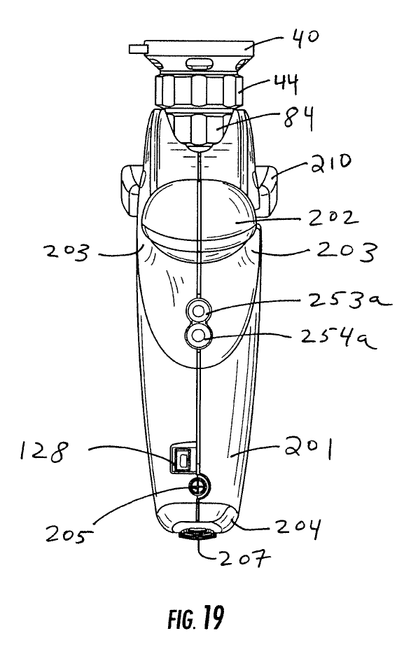

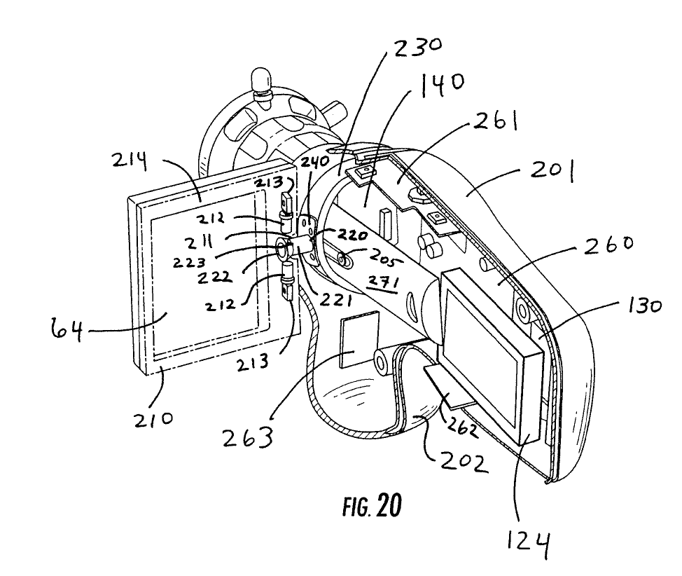

As best seen in FIGS. 20 and 21, display housing 210 and, in turn, video display 64, are coupled to main camera body 201 via display swivel post 220, display rotational connector 230, and display post connector 240. Display swivel post 220 comprises stem portion 221, swivel head portion 222, central bore 223, and a longitudinal axis extending through central bore 223. Swivel head portion 222 is retained within an interior region of display housing 210 by swivel hinge pins 212 which, in turn, are maintained in position by respective associated retention nuts 213. Stem portion 221 of display swivel post 220 extends from bottom aperture 211 of display housing 210 and into camera body 201 at display rotating slot 208 (as shown, for example, in FIG. 16). Electrical conductors, such as a ribbon cable, are passed through central bore 233 to electrically connect video display 64 to a printed circuit board carried within camera housing 200.

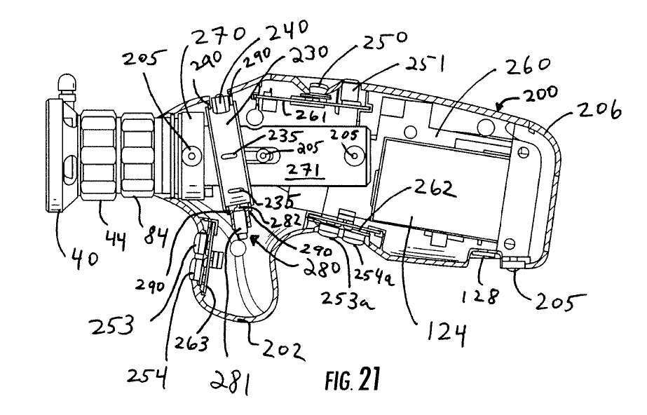

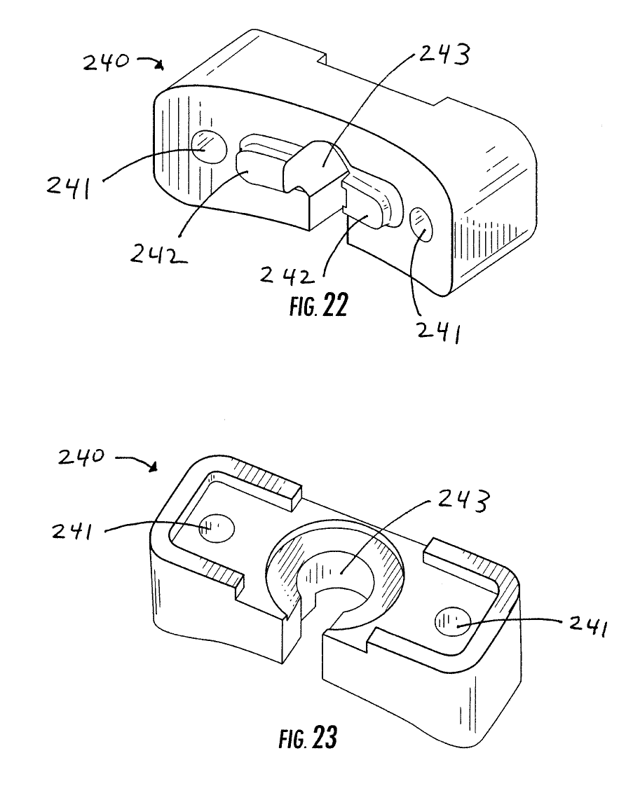

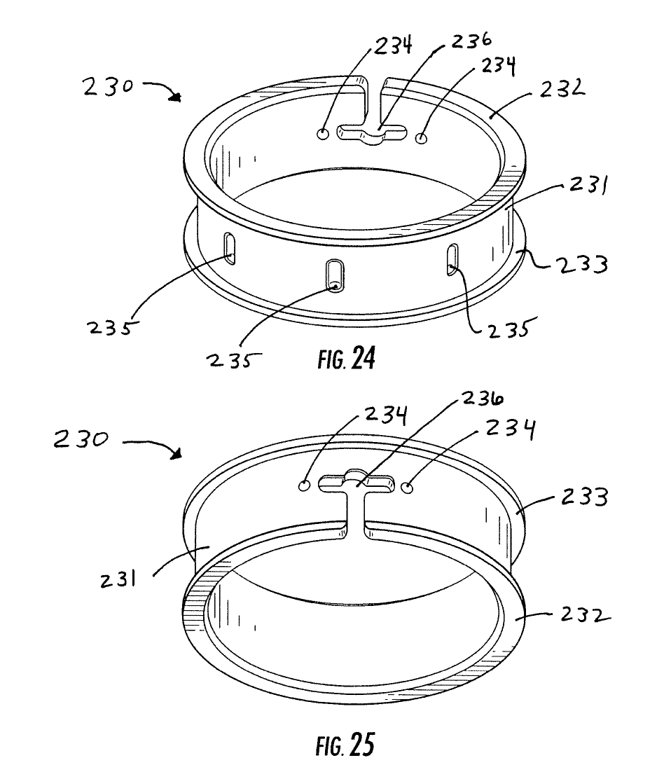

As shown in FIGS. 22-23, display post connector 240 includes two mounting apertures 241, locking members 242, and display swivel post stem accepting aperture 243. As shown in FIGS. 24-25, display rotational connector 230 comprises annular ring 231, first flanged end 232, second flanged end 233, display post connector mounting apertures 234, a plurality of detent apertures 235, and display swivel post stem aperture 235. Display post connector 240 secures stem portion 221 of display swivel post 220 to display rotational connector 230, with a distal end of stem portion 221 extending through swivel post stem aperture 235. Upon attachment of display post connector 240 and, in turn, display swivel post 220 to display rotational connector 230, locking members 242 are disposed within aperture 236, and respective mounting apertures 241 and 234 are aligned and secured together with suitable fasteners.

Upon attachment of display post connector 240 and display swivel post 220 to display rotational connector 230, stem portion 221 of the display swivel post extends outwardly from camera housing 200, through display rotating slot 208. As the display rotational connector is rotated back and forth, stem portion 221 travels back and forth through slot 208, through a range of motion limited by contact of stem portion 221 with slot endpoints 208a and 208b, disposed on the right and left hand sides of main body region 201, respectively, as shown in FIGS. 15 and 16. Moreover, and as shown in FIGS. 15 and 16, slot 208 extends farther down the left hand side of main body region 201, to endpoint 208b, than down the right hand side of main body region 201, to endpoint 208a. This permits the display housing to be secured for overall storage of the present endoscopic camera by first rotating the display counter-clockwise fully to the left (as viewed from the rear of camera housing 200), until stem portion 211 contacts endpoint 208b, and then rotating the display housing back towards battery door 206, until the display rests substantially flat against the left hand side of elongated main body region 201.

As shown in FIG. 20, display housing bottom aperture 211 includes an arcuate portion disposed through top face 214 of display housing 210. This, in turn, permits display housing 210 and display 64 not only to be rotated about the longitudinal axis of display swivel post 220 (as the two swivel hinge posts rotate about display swivel post 220 proximate the juncture of stem portion 221 and swivel head portion 222) but further permits display housing 210 and display 64 to be simultaneously pivoted towards and away from camera body 201, as stem portion 221 enters and exits the arcuate portion of bottom aperture 211, respectively. In a preferred embodiment, the range of pivotal movement of display housing 210, relative to camera body 201, extends from a first position, wherein display housing 210 is substantially perpendicular to a longitudinal axis of camera housing 200 (extending through the camera housing main body region 201, the center of zoom ring 84, and the center of focus ring 44), to a second position, where display housing 210 rests substantially flat against camera housing main body region 201.

As best seen in FIG. 21, display rotational connector 230 is rotatably carried within camera housing 200, with annular ring 231 substantially concentric to the longitudinal axis of camera housing 200. A plurality of webs, or bosses 290, integrally formed with and extending inwardly from the inner surface of camera housing 200, cooperate with first flanged end 232 and second flanged end 233 of display rotational connector 230, permitting annular ring to 231 to rotate about the longitudinal axis of the camera housing while retaining connector 230 in position within the housing.

As shown in FIG. 21, a click stop member 280, disposed within camera housing 200, includes a click stop member housing 281, and a spring biased pin 282, biased by a spring member in an extended orientation, relative to click stop member housing 281, and capable of coaxial back and forth motion, between extended and refracted positions, relative to click stop member housing 281. Spring biased pin 282 cooperates with the plurality of detent apertures 235 of display rotational connector 230, permitting display rotational connector 230 and, in turn, display housing 210, to be maintained in any of several click-stop orientations, all perpendicular to the longitudinal axis of the camera, as display rotational connector 230 and, in turn, display housing 210, is rotated about the longitudinal axis.

In particular, spring biased pin 282, when in its extended position, will releasably lock display rotational connector 230 in a particular degree of rotation, upon engagement with an associated detent aperture 235. While the spring biasing pressure placed upon pin 282 and is sufficient to maintain rotational connector 230 in a particular detent orientation, the spring pressure is not so strong so as to preclude the application of manual rotational force on display rotational connector 230 from causing spring biased pin 282 to retract sufficiently to permit further rotation of the connector, towards rotating the overall video display to another detent orientation.

As can be seen from the foregoing, display 64 and display housing 210 of the present endoscopic camera may be positioned in a wide variety of orientations, by rotating the display housing about two different axes of rotation, as well as pivoting the display about a pivot point spaced distally from the camera housing's main body portion. First, display housing 210 may be rotated, transverse to the longitudinal axis of display swivel post 220, about a point P1 (FIG. 15) spaced from the main camera body and proximate swivel head portion 222 of display swivel post 220, by rotating swivel hinge pins 212 about swivel head portion 222. Second, display housing may be pivoted about point P1, from a first position wherein display housing 210 is substantially aligned with stem portion 221 and is substantially perpendicular to main body region 201, towards the camera body to a second position where at least a portion of the display housing contacts main body region 201. Third, by rotating display rotational connector 230, point P1 and display housing 210 may be rotated along an arc defining a portion of a circle concentric to the longitudinal axis of camera housing 200, passing through main body region 201, focus ring 44 and zoom ring 84.

For example, FIG. 28 shows the present endoscopic camera being held relatively close to the body and relatively high in the air during an endoscopic inspection. For convenient viewing of display 64, display housing 210 is rotated about point P1 (FIG. 15) to substantially align the display with the physician's head (not shown), so that the physician's body need not be shifted or neck be craned in order to readily view an image displayed during the endoscopic inspection.

Additional internal components of the present endoscopic camera are shown in FIGS. 20-21 as including battery 124, removable flash memory card 130, image acquisition device 140, primary printed circuit board 260, secondary printed circuit boards 261, 262 and 263, coupler mount 270, and coupler mount extension 271. Battery 124 is preferably a conventional lithium-ion type battery, which may be removed for recharging in a separate charging unit by first removing battery door 206 from camera housing 200. Alternatively, or in addition, a battery recharging jack may be disposed on the surface of housing 200, and a suitable recharging cradle or stand supplied, to permit the battery to be recharged in situ.

Removable flash memory card 130 preferably comprises an industry standard Secure Digital (SD) card, Mini SD card with SD card adapter, or MultiMedia card (MMC). Memory card 130 is releasably retained within an associated card slot, and may be removed from within the camera housing upon removal of the battery door.

Primary printed circuit board 260 includes much of the circuitry depicted in FIG. 13, including A/D converter 112, digital signal processor or microprocessor 120, controller 122, and on-board flash memory 132. Secondary printed circuit board 261 carries direction button 250, mode button 251, and menu button 252. Secondary printed circuit board 262 carries redundant video record button 253a and redundant still photograph shutter button 254a. Secondary printed circuit board 263 carries redundant video record button 253 and redundant still photograph shutter button 254.

Electrical conductors, such as ribbon cables or flexible circuits, connect image acquisition device 140, secondary printed circuit boards 261, 262 and 263, and high speed USB data I/O port 128 to primary printed circuit board 260. Camera body 200 may further contain a miniature microphone (not shown), also coupled to primary printed circuit board 260. In conjunction with on-screen menu functions provided via display 64 and processor 120, the microphone permits the physician to record sound clips, such as voice annotations, to the internal flash memory storage or the removable flash memory card, and to transfer such sound/voice clips to an external personal computer.

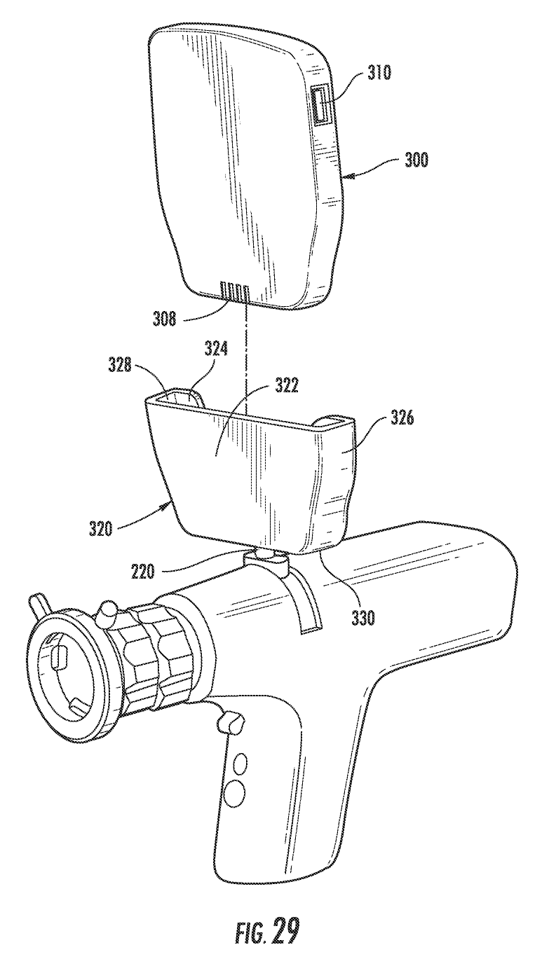

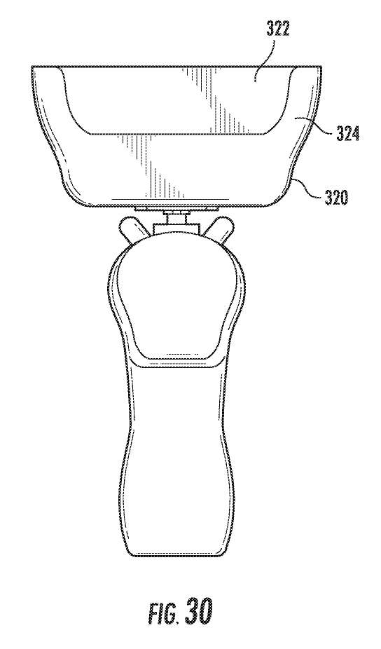

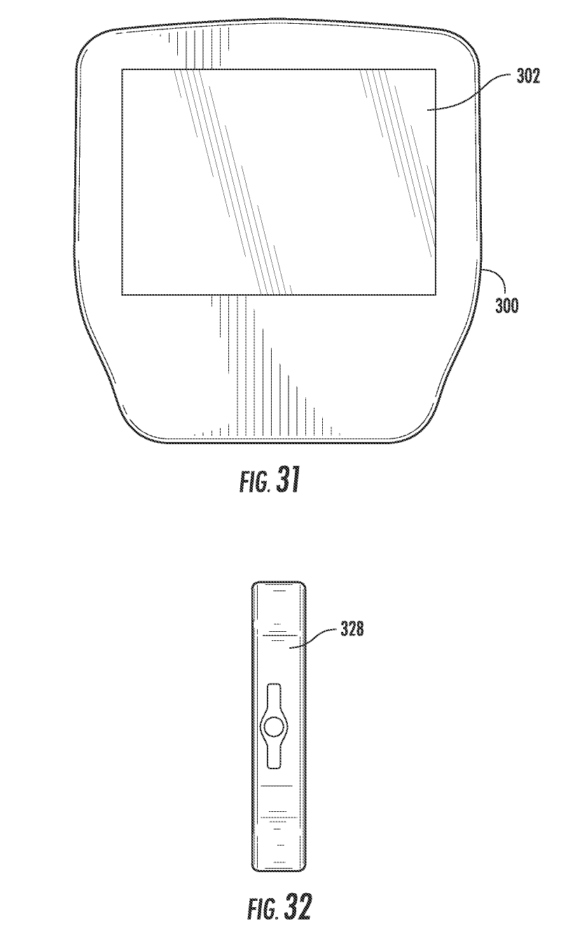

Referring now to FIGS. 29 through 32, there is shown an embodiment of a endoscopic imaging system with a removable hand-held image viewer or portable device 300 having a LCD screen 302. It is appreciated that said embodiment may include a High-Definition image sensor (1080i/1080p). It is further appreciated that the image viewer may act as a digital recording device as well. It is appreciated that the screen may be any of the known types of screens including, but not limited to, an LCD screen, LED screen or other similar monitor. The screen 302 may be a touch screen that permits a user to directly input information into the memory of the portable image viewer/recorder or to otherwise control the operation of the portable device. For example, using the portable device touch screen allows the user to tag an endoscopic exam to the patient's information without redundant effort. While a viewing screen is shown, it is appreciated that the portable device may not include a screen, wherein the image is transferred to a separate viewing device and may be shown on another screen associated with the image acquisition device/camera.

In one embodiment, the image viewer 300 includes flash RAM 304, or other internal memory, for storage of data as well as a slot for removable media such as a Secure Digital card 306 or similar storage. Data may further be transferred using known wireless and cellular transfer protocols. Connecting pins 308 located at the bottom of the image viewer are designed to engage corresponding pins of the cradle, docking station or other devices to permit the transferring of imaging data and other information. While connecting pins are shown, it is appreciated that other known connectors for interfacing with the camera or image acquisition device may be used. A power button 310 on the image viewer allows the image viewer to be selectively activated to preserve the battery life when not in use. Incorporated in the bottom of the device is a locking mechanism to allow for secure locking into cradles for the various configurations. Examples of locking mechanism include, but are not limited to, detents or interior walls that permit the image viewer to be press fit into the cradle.

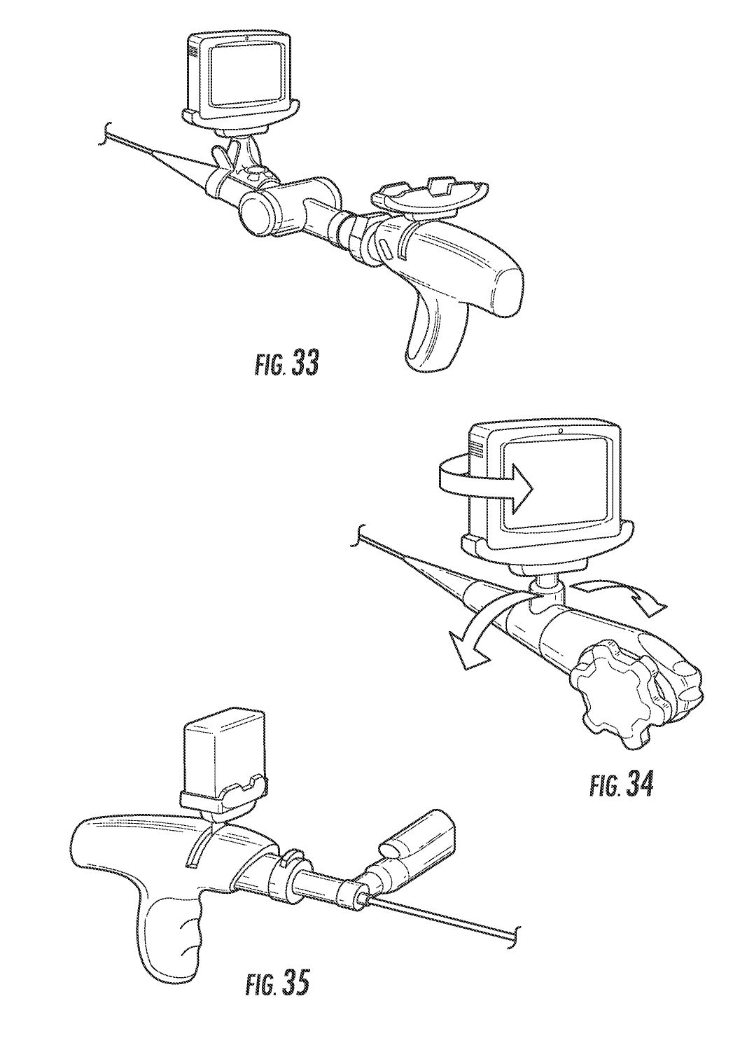

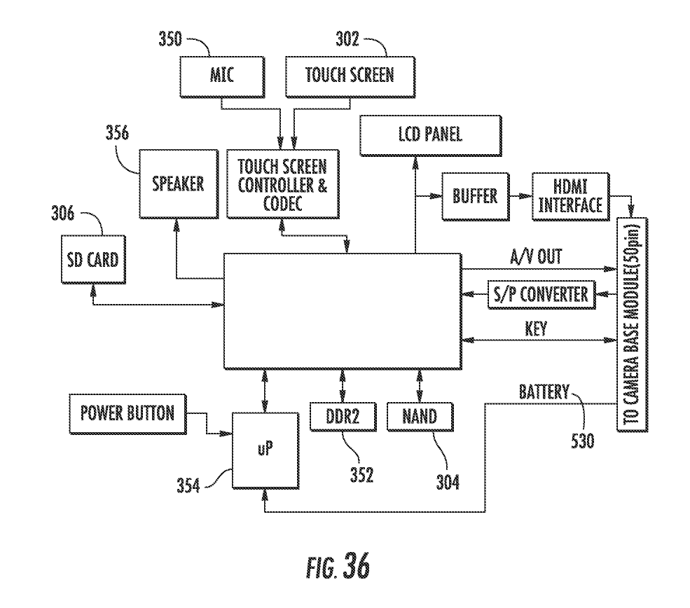

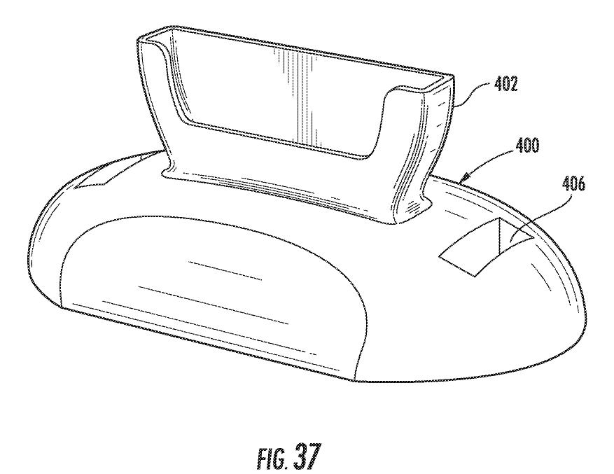

As shown in FIG. 29, the image viewer 300 is sized and shaped to removable fit within a corresponding cradle 320 of a hand held camera for use with an endoscope. While FIG. 29 shows the use of a cradle for a flexible optical endoscope, it is appreciated that the image viewer may be used with any known endoscopes by attaching to, among other things: a cradle for use with a trigger mechanism (FIG. 33) which allows for more ergonomic positioning of the LCD touch screen for better viewing; a cradle on a hand-held camera for use with a distal chip endoscope (FIG. 34); or a cradle on a hand-held camera for use with an optical rigid scope (FIG. 35). Referring again to FIG. 29, it is appreciated that any configuration of optical scopes can be used in this manner (nasopharyngoscopes, GI scopes, bronchscopes, etc.).

The cradle 320 includes a rear wall 322, front wall 324, side walls 326, 328 and a bottom 330 that define a cavity for receiving the bottom portion of the image viewer 300. Pins (not shown) on the inside of the image viewer are located to engage or interface with the corresponding pins 308 of the image 300 viewer to provide a link to allow images and other data to be exchanged between the image viewer and camera. It is appreciated that other known connectors may be used to interface with the image viewer. The front wall 324 of the cradle 320 may be cut away to permit a larger screen 302 to be viewed in its entirety while still being secured in the cradle 320. In order to prevent the accidental separation of the image viewer from the cradle, it is appreciated that a locking or securing mechanism may be employed. It is appreciated that any of the known methods for securing a portable device in a cradle may be used including, but not limited to, utilizing a press fit or releasable detents.

The image viewer may also include a gravity detection device and user enabled software programming to orientate the images on the viewing screen with respect to the detected gravity reference. In a preferred embodiment, the image viewer/recorder provides a high definition system, and includes lighting controls, full multi-media system, on-board data management and integration programming. LED lighting technology may be provided for brighter lighting capability than in many tabletop Halogen or Metal Halide light sources.

The image viewer is preferably constructed from high impact plastic, although other known sturdy materials also may be used. Similar to the connection of the integral image viewer or display housing 210 set forth above, it is appreciated that the cradle may be attached to the main camera body 201 via swivel post 220, display rotational connector 230, and display post connector 240. It is further appreciated that the cradle may be attached to the main camera body or endoscopic system in other known ways including, but not limited to, a rigid connection, and not depart from the scope of the present invention.

FIG. 36 is a schematic block diagram of one embodiment of the portable device of the present invention. Information or data may be inputted through the touch screen 302 and/or a sound port (“MIC”) 350, while audio information and data may be played through speaker 356. The portable device 300 may utilize internal memory such as double data rate RAM 352, or through an SD card 306 or a NAND gate or flash memory device 304. A power button 310, which may be located on the side of the portable device 300, communicates with the microprocessor 354 to selectively activate the portable device 300.

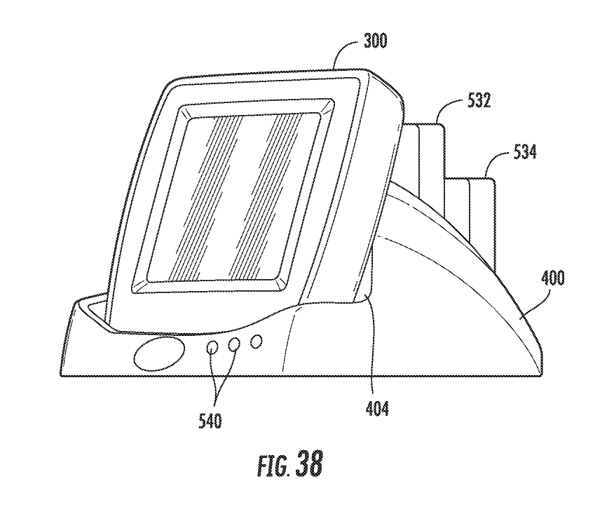

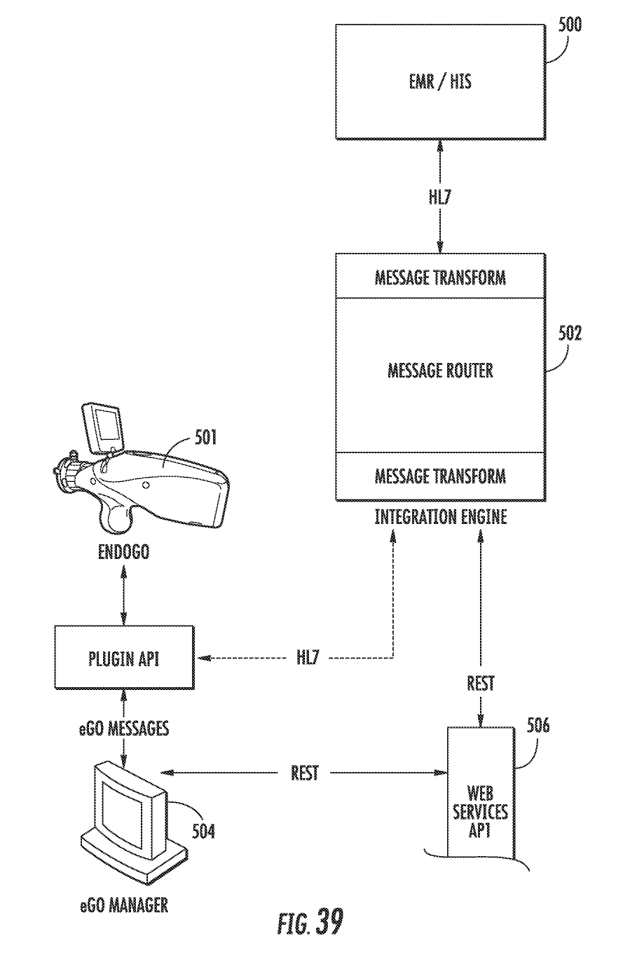

Referring to FIGS. 37 and 38, examples of docking stations 400 that may be used in connection with the portable device or image viewer are shown. While docking stations 400 utilizing a cradle 402 (FIG. 37) or front compartment 404 (FIG. 38) are shown and disclosed, it is appreciated that the portable device may be used with other docking stations and not depart from the scope of the present invention. The docking station 400 contains a digital/analog converter and printed circuit boards to allow for appropriate signal conversion (I/O). Further, in one embodiment, the docking station 400 contains various inputs/outputs 406 such as USB, IEEE, RGB, BNC, composite, S-video, HDMI, and Ethernet to permit the docking station to securely exchange data with other devices, such as a network computer, or a hospital information system (“HIS”)/EMR system in a secure format (e.g., in compliance with health level 7 (“HL7”) standard). A power cable such as an AC power adaptor and cord (not shown) attaches to a power input to permit power to be supplied to the docking station. The docking station also preferably contains a mechanism to synchronize information exchange between the portable device and a computer or other operating system.

FIG. 39 represents a schematic illustration of one embodiment of use of the portable device 300 with a hospital information system and/or electronic medical records (“EMR”) 500. Particularly, the hand-held camera 501 transfers images taken to the portable device 300 (referred to as an eGo), which may then securely transmit information to a patient's electronic medical record or hospital information system 500 through a message router 502. The data or information being transmitted may be transformed in a known way to permit it to be integrated or synchronized with the information in the EMR/HIS. The information may be transmitted from a computer or system 504 connected to the portable device, or if the portable device is Internet capable, through the Internet using Representative State Transfer (“REST”) via a web services API 506 or other known means. It is appreciated that the computer or system connected to the portable device includes software that permits a user to manage, edit, convert or otherwise prepare the information for transmission to an HIS/EMR or other system or user.

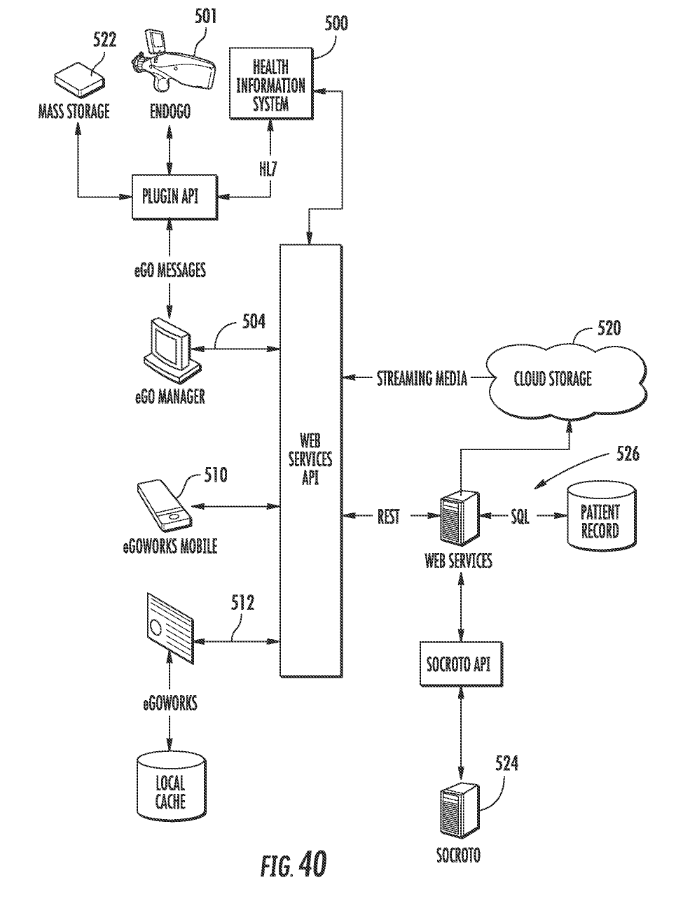

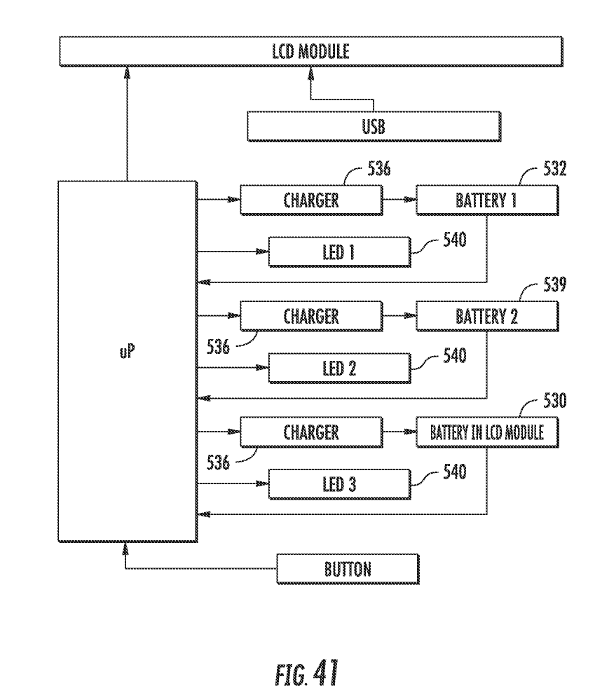

While a computer managing system is shown in FIG. 39, as shown schematically in FIG. 40, it is appreciated that a user also may manage the information or data remotely from, among other things, a mobile device 510, other computing systems, or through the Internet 512. In order to permit the information and data transmitted from the endoscope or otherwise inputted to be accessed from any location, the information and data may be stored in a cloud storage database 520. Alternatively, the information and data may be stored in a local or portable mass storage device 522. The system may also permit communication with other physicians 524 to exchange ideas or questions through the Internet 526 (e.g., through the Socroto.com website).

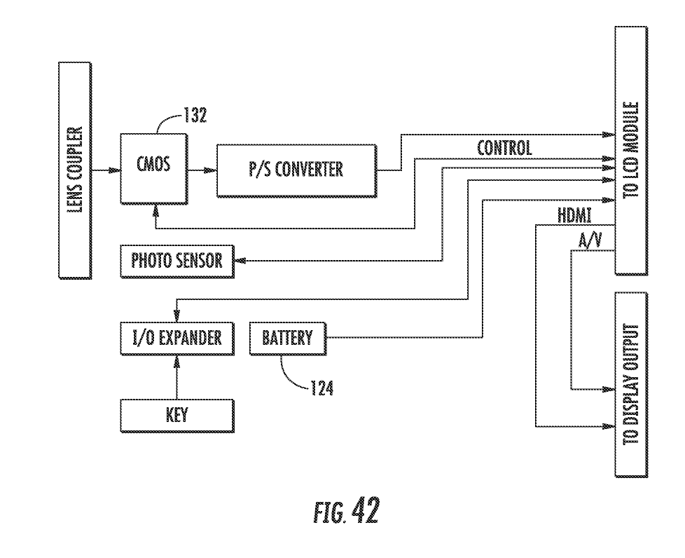

Referring to FIGS. 38 and 41, the docking station may also permit the battery 530 of the portable device 300 and extra batteries 532,534 to be recharged by inserting them in respective slots 536 or compartments in the docking station 400. LED lights or other indicators 540 may be utilized to graphical display the status of the charging of the batteries 530,532,534. For example, a red LED light may indicate that the battery is still charging while a green LED light may indicate that the battery is fully charged and may be removed for use.

One embodiment of the portable device may be used as a universal storage device for various endoscopic technologies such as optical endoscopes and camera, distal chip camera systems and DICOM image viewers. Images can be stored in a wide-variety of formats including, but not limited to, in JPEG photo format, in various movie formats (MPEG, .AVI, .MOV, etc.), and as DICOM images. In this configuration, images are acquired from the endoscope (optical or distal chip) and stored in memory (e.g., on the integrated flash RAM or removable SD card) on the portable device. Once stored in memory, the images can be viewed directly from the portable device.

The portable device can also can be affixed to a docking station for output via various means (HDMI, composite, USB, BNC) to an array of output devices (CRT monitors, LCD/Plasma/LED monitors, computer monitors). Further, wireless transmission via BlueTooth/80211.b/High Definition/Cellular and similar methods of wireless transmission can be used to transmit the data to HIS/EMR or other systems or computers. Transmission via a cellular network for live streaming video and data may also be utilized. HD wireless transmission may allow for live (full motion) uncompressed data transfer to a slave monitor capable of receiving such signal.

The portable device may also be used in connection with legacy cart based endoscopic systems. In this configuration, various input methods from the legacy cart system are used to transmit/transfer images to the portable device (composite, BNC, HDMI, etc.). Once the images are stored, viewing can be done on the portable device or on various viewing devices (CRT, LCD/Plasma/LED monitors, computer monitor).

Where data and information is transmitted or otherwise communicated with an EMR or HIS, it is appreciated that the images and other data and information are synced for complete integration of imaging from the point of care to storage and management. The advantage of this approach is to remove redundant/repetitive tasks associated with current acquisition of endoscopic imaging data which usually requires user input to rename and catalogue files.

In another embodiment, it is appreciated that the data and information and data from the portable device may be transmitted for storage in an online database or data management site. This method of data storage and management streamlines storage and management to facilitate viewing in multiple locations via the internet. In practice environments without LANs or a central server, this can facilitate patient care.

The portable device may also be used to transmit video/images and data over a cellular network or other wireless network, which can provide a unified medical communications network for easy, instant access to medical information. In this embodiment, either during the live recording or after the recording is stored, the data can be transmitted over a cellular network employing GSM and/or CDMA technology with 3G and/or 4G (or equivalent bandwidth) to remotely transmit the images/data.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention. For example, it is also anticipated that the viewing screen on the camera may be a commercially available twin LCD display having a backlight and a system LSI (large-scale integrated circuit) chip between two LCD screens, allowing both sides of the display to work at the same time. Further, the system may include an audio input for accommodating stroboscopic analysis.

1. A medical image recording and viewing system for use with endoscopes having an image acquisition device, the medical recording and viewing system comprising:
a portable hand-held device comprising:
a housing;
a power source;
a first connector;
a processor;
a viewing screen; and
memory for processing data from the image acquisition device and for displaying at least one image on the viewing screen; and
a cradle attached to at least one of the endoscopes, wherein the cradle is operatively attached to the image acquisition device, and includes a second connector to create an interface between the image acquisition device and the portable hand-held device.

2. The medical image recording and viewing system of claim 1, wherein the viewing screen is an LCD screen.

3. The medical image recording and viewing system of claim 1 wherein the viewing screen is a touch screen.

4. The medical image recording and viewing system of claim 1 wherein the portable hand-held device also receives and stores data.

5. The medical image recording and viewing system of claim 1 which further comprises a lock for securely retaining the portable hand-held device within the cradle.

6. The medical image recording and viewing system of claim 1 wherein the portable hand-held device further comprises a gravity detection device.

7. The medical image recording and viewing system of claim 1 wherein the housing of the portable hand-held device is made from a high-impact plastic.

8. The medical image recording and viewing system of claim 1 wherein the portable hand-held device further comprises a wireless transmitter.

9. The medical image recording and viewing system of claim 1 which further comprises a docking station having a third connector for interfacing with the first connector of the portable hand-held device, and a power supply.

10. The medical image recording and viewing system of claim 9 wherein the power source of the portable hand-held device is a battery, and wherein the docking station further comprises a battery charger for recharging the battery.

11. The medical image recording and viewing system of claim 9 wherein the docking station further comprises an input/output connection for connecting to other electrical devices.

12. The medical image recording and viewing system of claim 10 wherein the docking station further includes an indicator for providing the status of the charging of the battery.

13. The medical image recording and viewing system of claim 1, wherein the recorder is synchronized to an electronic medical record or other source of patient data.

14. The medical image recording and viewing system of claim 1, wherein the cradle is affixed to a hand-held endoscopic camera.

15. The medical image recording and viewing system of claim 1, wherein the cradle is affixed to a distal chip endoscope.

16. The medical image recording and viewing system of claim 1, where the at least one image is digital video.

17. A method of capturing, archiving and sharing endoscopic images, the method comprising the steps of:
providing a portable device capable of coupling to a portable hand-held endoscopic camera and scope;
storing at least one of the images from an endoscopic examination in real time to a memory device of the portable device;
simultaneously displaying the at least one of the images on a viewing screen of the portable device; and
subsequently coupling the portable device to another device for further processing.

18. The method of claim 17 which further comprises the step of downloading a user's schedule to the portable device.

19. The method of claim 17 which further comprises the step of synchronizing the data between the portable device and a storage device.

20. A medical image recording system for use with endoscopes having an image acquisition device, the medical recording and viewing system comprising:
a portable hand-held device comprising:
a housing;
a power source;
a first connector;
a processor; and
memory for processing data from the image acquisition device; and
a cradle attached to at least one of the endoscopes, wherein the cradle is operatively attached to the image acquisition device, and includes a second connector to create an interface between the image acquisition device and the portable hand-held device.

21. The medical image recording system of claim 20 which further comprises a docking station having a third connector for interfacing with the first connector of the portable hand-held device, and a power supply.

22. The medical image recording system of claim 20 wherein the docking station further comprises an input/output connection for connecting to other electrical devices.

23. A medical image recording and viewing system comprising:
an endoscope having an image acquisition device;
a cradle attached to the endoscope and operatively attached to the image acquisition device, the cradle defining a cavity; and
a portable hand-held device for removable placement within the cavity of the cradle, wherein the portable hand-held device includes a viewing screen and a memory for storing images received from the image acquisition device, and wherein the images may be displayed on the video screen.
